(19) United States
(12) Patent Application Publication
(10) Pub. No.: US 2004/0133036 A1
(43) Pub. Date: Jul. 8, 2004

(54) Method for producing hydroxyalkyl carboxylic acid esters

(76) Inventors: Michael Zirstein, Schriesheim (DE); Georg Heinrich Grosch, Bad Durkheim (DE); Edward Bohres, Ludwigshafen (DE); Alfred Oftring, Bad Durkheim (DE); Stefan Birnbach, Dirmstein (DE); Werner Bochnitschek, Ludwigshafen (DE); Oliver Borzyk, Speyer (DE); Rainer Corell, Worms (DE); Harald Wurz, Edenkoben (DE); Thomas Heidemann, Viernheim (DE); Marcus Sigl, Mannheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/474,147

(22) PCT Filed: Apr. 5, 2002
(86) PCT No.: PCT/EP02/03819

(30) Foreign Application Priority Data
Apr. 6, 2001 (DE) 10117273.7
Nov. 8, 2001 (DE) 10154781.1

(51) Int. Cl. C07C 249/00

(52) U.S. Cl. 560/336

(57) ABSTRACT

A process for preparing hydroxyalkyl carboxylates comprises reacting a carboxylic acid with an alkylene oxide in the presence of a multimetal cyanide compound of the formula I

M³_{Z}M¹ₐ[M²(CN)_{b}(A)_{c}]_{d}·fM¹_{g}Xₙ·mM³ₚY_{q} ·h(H₂O)·eL·kP (I)

preferably applied to a solid support or shaped to form a shaped body, as catalyst. The hydroxyalkyl carboxylates prepared in this way can be used as raw materials for surface coatings.

The present invention relates to a process for preparing hydroxyalkyl carboxylates from carboxylic acids and alkylene oxides using a multimetal cyanide compound, preferably applied to a solid support or shaped to form a shaped body, as catalyst for the reaction, and also to the use of the hydroxyalkyl carboxylates prepared according to the present invention as raw materials for surface coatings.

Processes for preparing hydroxyalkyl carboxylates in the presence of catalysts are known per se. The type of catalysts used varies widely.

For example, DE 1 248 660 describes a process for preparing glycol monoesters from carboxylic acids and alkylene oxides in the presence of thioethers.

U.S. Pat. No. 4,970,333 describes the preparation of esters by reaction of an epoxide with carboxylic acid in the presence of a strongly basic ion exchange resin. In particular, acrylic resins are used.

U.S. Pat. No. 4,910,329 relates to a process for preparing hydroxyalkyl esters of acrylic and methacrylic acids in the presence of heterogeneous amorphous catalysts, the catalysts used here being metal phosphates.

DE 1 255 104 describes a process for preparing β-hydroxyalkyl monoesters of acrylic and methacrylic acids in the presence of iron acrylate or iron methacrylate catalysts.

According to U.S. Pat. No. 3,059,024, β-hydroxyalkyl monoesters of acrylic and methacrylic acids can also be prepared from carboxylic acid and ethylene oxide or propylene oxide using tetraalkylammonium salts as catalyst.

U.S. Pat. No. 2,484,487 describes a process for preparing glycol monoesters which comprises reacting an alkylene oxide with acrylic or methacrylic acid in the presence of a tertiary amine as catalyst.

The known processes suffer from a series of disadvantages, for example unsatisfactory conversions, unsatisfactorily low selectivity, polymerization of the carboxylic acid when unsaturated carboxylic acids are employed, and also secondary reactions during the work-up of the products. In addition, volatile catalyst components sometimes have to be used and the catalyst-containing distillation residues can be disposed of only with difficulty.

Metal cyanide compounds are known from the prior art as catalysts for polyadditions, in particular for ring-opening polymerizations of alkylene oxides, as described, for example, in EP-A 0 892 002, EP-A 0 862 977 and EP-A 0 755 716. WO 99/16775 describes multimetal cyanide catalysts which can be used, in particular, for the alkoxylation of compounds containing active hydrogen.

The preparation of polyether alcohols using supported double metal cyanide catalysts is described, for example, in WO 99/44739.

WO 99/10407 describes a process for preparing polyethers having a hydroxy functionality and containing unsaturated groups. The synthesis is carried out by alkoxylation of an unsaturated monomer containing reactive hydrogen atoms. The reaction is carried out in the presence of a double metal cyanide catalyst whose preparation is described in more detail in U.S. Pat. No. 5,545,601. In the process described there, unsaturated carboxylic acids can also be used as monomers in the reaction with an alkylene oxide.

WO 99/10407 concerns the synthesis of multiple alkoxylation products. Polyethers and not the corresponding monoalkoxylation products of the starter molecules are obtained using the catalysts described in the examples. Further alkoxylation takes place more readily than the monoalkoxylation, so that the monoalkoxylation product is not obtained selectively.

It is an object of the present invention to provide suitable catalysts for the reaction of carboxylic acids with alkylene oxides, by means of which the desired hydroxyalkyl carboxylates, i.e. the monoalkoxylation products, can be prepared with high selectivity and without formation of undesirable by-products.

We have found that this object is achieved by a process in which a multimetal cyanide compound of the formula I is used as catalyst.

The present invention accordingly provides a process for preparing hydroxyalkyl carboxylates, which comprises reacting at least one carboxylic acid with at least one alkylene oxide in the presence of a multimetal cyanide compound of the formula I as catalyst:

M³_{Z}M¹ₐ[M²(CN)_{b}(A)_{c}]_{d}·fM¹_{g}Xₙ·mM³ₚY_{q} ·h(H₂O)·eL·kP (I)

where

M¹ is at least one metal ion selected from the group consisting of Zn²⁺, Fe²⁺, Fe³⁺, Co³⁺, Ni²⁺, Mn²⁺, Co²⁺, Sn²⁺, Pb²⁺, Mo⁴⁺, Mo⁶⁺, Al³⁺, V⁴⁺, V⁵⁺, Sr²⁺, W^{4+,} W⁶⁺, Cr²⁺, Cr³⁺, Cd²⁺, Hg²⁺, Pd²⁺, Pt^{2 +}, Mg²⁺, Ca²⁺, Ba²⁺, Cu²⁺,

M² is at least one metal ion selected from the group consisting of Fe²⁺, Fe³⁺, Co²⁺, Co³⁺, Mn²⁺, Mn³⁺, V⁴⁺, V⁵⁺, Cr²⁺, Cr³⁺, Rh³⁺, Ru²⁺, Ir³⁺,

M¹ and M² are identical or different and at least M¹ or M² is Fe²⁺or Fe³⁺,

M³ is at least one metal ion selected from the group consisting of Lⁱ⁺, Na⁺, K⁺, Rb⁺, Cs⁺, Mg²⁺, Ca²⁺, Ba²⁺, Sr²⁺, ammonium ions of the formula R¹R²R³R⁴N⁺, where R¹, R², R³ and R⁴ are each H or a hydrocarbon radical having from 1 to 6 carbon atoms,

A, X and Y are each, independently of one another, an anion selected from the group consisting of halide, hydroxide, sulfate, carbonate, cyanide, thiocyanate, isocyanate, cyanate, carboxylate, oxalate, nitrate, nitrosyl, hydrogensulfate, phosphate, dihydrogenphosphate, hydrogenphosphate and hydrogencarbonate,

L is a water-miscible ligand selected from the group consisting of alcohols, aldehydes, ketones, ethers, polyethers, esters, polyesters, polycarbonate, ureas, arnides, primary, secondary and tertiary arnines, ligands containing pyridine nitrogen, nitriles, sulfides, phosphides, phosphites, phosphines, phosphonates and phosphates,

k is a fraction or integer greater than or equal to zero, and

P is an organic additive,

a, b, c, d, g, n, p, q and z are selected so that the compound (I) is electrically neutral, where c or z or c and z may be 0,

e is the number of ligand molecules and is a fraction or integer greater than 0 or is 0,

f, k, h and m are each, independently of one another, a fraction or integer greater than 0 or are 0.

Organic additives P which may be mentioned are: polyethers, polyesters, polycarbonates, polyalkylene glycol sorbitan esters, polyalkylene glycol glycidyl ethers, polyacrylamide, poly(acrylamide-co-acrylic acid), polyacrylic acid, poly(acrylamide-co-maleic acid), polyacrylonitrile, polyalkyl acrylates, polyalkyl methacrylates, polyvinyl methyl ether, polyvinyl ethyl ether, polyvinyl acetate, polyvinyl alcohol, poly-N-vinylpyrrolidone, poly(N-vinylpyrrolidone-co-acrylic acid), polyvinyl methyl ketone, poly(4-vinylphenol), poly(acrylic acid-co-styrene), oxazoline polymers, polyalkylenimines, copolymers of maleic acid and maleic anhydride, hydroxyethylcellulose, polyacetates, ionic surface-active and interface-active compounds, bile acids or their salts, esters or amides, carboxylic esters of polyhydric alcohols and glycosides.

The process of the present invention makes it possible to prepare hydroxyalkyl carboxylates from carboxylic acids and alkylene oxides in high purity and with high selectivity and without further alkoxylation of the products occurring, by means of matching of the temperature and pressure to an appropriate catalyst system.

According to the present invention, it is also possible for the multimetal cyanide compound to be applied to a solid support or be shaped to form a shaped body. The use of a catalyst which has been shaped to form a shaped body or been supported enables the catalyst to be removed in a simple manner from the reaction mixture after the reaction or to be immobilized in a fixed bed.

In a preferred embodiment, the present invention accordingly provides a process for preparing hydroxyalkyl carboxylates in which the multimetal cyanide compound has been applied to a solid support or been shaped to form a shaped body.

For the purposes of the present invention, a solid support is a macroscopic shaped body. Suitable supports and the production of multimetal cyanide catalysts which have been supported or been shaped to form shaped bodies are described, for example, in WO 99/44739, which is hereby fully incorporated by reference.

For the purposes of the present invention, a shaped body is a three-dimensional macroscopic body.

The shaped bodies used according to the present invention can have any shape, for example pellets, annular pellets or extrudates, in particular round, hollow or star-shaped extrudates. The dimensions of the shaped bodies used according to the present invention can vary. For the purposes of the present invention, preference is given to shaped bodies having a length of from 1 to 20 mm, in particular from 3 to 10 mm, and a diameter of from 1 to 10 mm, in particular from 1.5 to 5 mm.

Possible supports are, for example, extrudates, granules, pellets, gauzes, packing elements, woven fabrics, fibers, spheres and the interior walls of reactors. The macroscopic shaped bodies can comprise inorganic or organic materials. Inorganic materials are, for example, oxides, carbides, nitrides or inert metals. Examples of carbides are transition metal carbides such as tungsten carbide, and also silicon carbide and boron carbide. Suitable nitrides are, for example, boron nitride, silicon nitride or aluminum nitride. Inert metals are, for the purposes of the present invention, metals or metal alloys which are inert toward the reaction medium of the synthesis of the multimetal cyanide compound and display inert behavior in the hydroxyalkyl carboxylate synthesis. Examples are steels, aluminum, noble metals, nickel, stainless steels, titanium, tantalum and Kanthal. As oxides, it is possible to use metal oxides which are inert under the conditions of the reaction, in particular oxides of metals of groups IIa to IVa and Ib to VIIIb, and also oxidic compounds which contain elements of groups Ia to VIIa or metals of groups Ib to VIIIb.

Such catalysts can be produced by applying multimetal cyanide compounds to the surface of the shaped supports or by mixing multimetal cyanide compounds with unshaped support materials and subsequently shaping the mixture. For the purposes of the present invention, it is also possible to shape pulverulent multimetal cyanide compounds to give all-active catalysts.

This can be done by tableting or extrusion. In tableting, it is generally necessary to add lubricants. These can be graphite, boron nitride or organic molecules such as stearates or alginates. The multimetal cyanide compounds are not heated to above 200° C., preferably not above 150° C., after the shaping step.

For the purposes of extrusion, the multimetal cyanide compounds are firstly compounded with a make-up liquid in a kneader, pan mill or similar apparatus to produce a plastic composition. In this kneading step, further ingredients can be added to the composition being formed so as to improve the properties of the plastic composition in the actual shaping step or to give the shaped body produced from this composition better cohesion or to produce variations in respect of pore volume and pore gradient distribution. In principle, all suitable additives known to those skilled in the art can be used. When adding the additives, particular care has to be taken to ensure that they display their desired action, for example promotion of cohesion or formation of porosity, in a heat treatment step carried out subsequent to shaping at not more than 200° C., preferably not more than 150° C. Furthermore, the additives should not reduce the catalytic activity of the multimetal cyanide compounds. The amount of additives is selected so that they display their full action but the content is not so high that the catalytic activity of the multimetal cyanide compounds is reduced.

Such supported multimetal cyanide compounds or multimetal cyanide compounds shaped to form shaped bodies can likewise be used in the process of the present invention and, like the pulverulent catalyst, lead with high selectivity to the monoalkylation product in the reaction of an unsaturated carboxylic acid with an alkylene oxide. This is surprising because, compared to the use of a pulverulent catalyst, the shaping or supporting of the multimetal cyanide compound can result in problems in the transport of starting materials or products to or from the active catalyst surface as a result of longer diffusion paths, so that the product selectivity to the monoalkoxylation product should be adversely affected since subsequent reactions are said to occur preferentially (cf., for example, J. Hagen, Technische Katalyse, VCH Verlagsgesellschaft, Weinheim, 1996, p. 87 ff.).

In a further embodiment, the invention provides a process for preparing hydroxyalkyl carboxylates in which one or more of the following conditions are fulfilled:

(A) M¹ is selected from the group consisting of Zn²⁺, Fe²⁺, Fe³⁺, Co³⁺, Ni²⁺, Mn²⁺, Co²⁺;

(B) M² is selected from the group consisting of Fe²⁺, Fe³⁺, Co³⁺;

(C) M³ is selected from the group consisting of Na⁺, K⁺, ammonium ions of the formula R¹R²R³R⁴N⁺;

(D) M¹ or M² is Fe²⁺or Fe³⁺.

In a further preferred embodiment of the present invention, both M¹ and M² are Fe²⁺ or Fe³⁺, in particular together with the further preferred metal ions mentioned under (A) to (C).

Catalysts which have been found to be particularly useful for the purposes of the present invention are, for example, the following multimetal cyanide compounds: iron blue pigments, iron cyanide blue, Vossen-Blau®, Prussian blue, Berlin blue, Turnbull's blue, Milori blue, Paris blue.

The use according to the present invention of the multimetal cyanide compounds in which at least M¹ or M² is Fe²⁺ or Fe³⁺ enables the desired monoalkoxylation products to be prepared with high selectivity. If multimetal cyanide catalysts containing no Fe²⁺ or Fe³⁺ are used under otherwise identical reaction conditions, multiple alkoxylation occurs.

The multimetal cyanide compounds are generally produced by reaction of at least one metal salt with at least one cyanometallic compound. As cyanometallic compound, it is possible to use, for example, salts or acids. In the process of the present invention, the presence of alkali metal salts or alkaline earth metal salts as impurities does not interfere, so that complicated and costly purification of the catalysts is unnecessary.

In a preferred embodiment, the invention provides a process for preparing hydroxyalkylcarboxylates in which the multimetal cyanide compound used as catalyst is crystalline or partly amorphous.

In the process of the invention, it is possible for a catalyst precursor compound to be prepared first and then be converted into the actual catalytically active compound, for example by means of oxidation, reduction, recrystallization or other reactions. It is thus also conceivable for the purposes of the present invention for, for example, the precursor compound to be used in the reaction and for the actual catalytically active compound to be formed only in the reaction medium in the presence of the components to be reacted.

It is also possible for the morphology of the multimetal cyanide particles to be controlled by addition of suitable substances, for example surface-active substances, so that an increased activity for the reaction to be catalyzed is achieved.

In the process of the present invention, the amount of catalyst used is from 0.001 to 30% by weight, preferably from 0.01 to 10% by weight, particularly preferably from 0.1 to 5% by weight or from 0.2 to 3% by weight, in each case based on the amount of carboxylic acids used.

The invention therefore provides, in particular, a process for preparing hydroxyalkyl carboxylates in which the catalyst is used in amounts of from 0.01 to 30% by weight, based on the amount of carboxylic acid used.

In principle, all substituted and unsubstituted, branched or unbranched carboxylic acids can be used in the process of the present invention as long as the functional groups of the carboxylic acid do not adversely affect the catalyzed reaction.

For the purposes of the present invention, preference is given to using substituted or unsubstituted, saturated or unsaturated monocarboxylic acids having from 3 to 22 carbon atoms, substituted or unsubstituted, saturated dicarboxylic acids having from 2 to 36 carbon atoms, substituted or unsubstituted, unsaturated dicarboxylic acids having from 4 to 36 carbon atoms and substituted or unsubstituted aromatic monocarboxylic and dicarboxylic acids.

Accordingly, the invention provides, in a further embodiment, a process for preparing hydroxyalkyl carboxylates in which the carboxylic acid is selected from the group consisting of substituted or unsubstituted, saturated or unsaturated monocarboxylic acids having from 3 to 22 carbon atoms, substituted or unsubstituted, saturated dicarboxylic acids having from 2 to 36 carbon atoms, substituted or unsubstituted, unsaturated dicarboxylic acids having from 4 to 36 carbon atoms and substituted or unsubstituted aromatic monocarboxylic and dicarboxylic acids.

In particular, the following carboxylic acids may be mentioned as preferred according to the present invention: unsaturated substituted or unsubstituted monocarboxylic acids having from 3 to 5 carbon atoms and unsaturated substituted or unsubstituted dicarboxylic acids having from 4 to 8 carbon atoms, for example acrylic acid, methacrylic acid or crotonic acid, fumaric acid, maleic acid or itaconic acid; saturated substituted or unsubstituted monocarboxylic acids having from 1 to 5 carbon atoms and saturated substituted or unsubstituted dicarboxylic acids having from 2 to 5 carbon atoms, for example formic acid, acetic acid, propionic acid, pivalic acid, oxalic acid, malonic acid or succinic acid; saturated or unsaturated substituted or unsubstituted monocarboxylic acids which have from 6 to 22 carbon atoms and may also contain cycloaliphatic structural elements, for example hexanoic acid, heptanoic acid, cyclohexanecarboxylic acid, 2-ethylhexanoic acid, capric acid (C10), myristic acid (C14), palmitic acid (C16), stearic acid (C18), oleic acid, behenic acid (C22); saturated or unsaturated substituted or unsubstituted dicarboxylic acids which have from 6 to 36 carbon atoms and, in particular, contain cycloaliphatic structural elements, for example adipic acid, pimelic acid (C7), azelaic acid (C9), sebaccic acid (C10), dimeric fatty acids having 36 carbon atoms; substituted or unsubstituted aromatic monocarboxylic and dicarboxylic acids, for example benzoic acid, phthalic acid, isophthalic acid, terephthalic acid or naphthalenecarboxylic acids.

Particular preference is given to using acrylic acid and methacrylic acid. The invention therefore provides, in a preferred embodiment, a process for preparing hydroxyalkyl carboxylates in which the carboxylic acid is acrylic acid or methacrylic acid.

In the process of the present invention, it is in principle possible to use all alkylene oxides which are known to those skilled in the art. In particular, use is made of substituted or unsubstituted alkylene oxides having from 2 to 24 carbon atoms, in particular alkylene oxides bearing halogen, hydroxy, acyclic ether or ammonium substituents. Particular mention may be made of: aliphatic 1,2-alkylene oxides having from 2 to 4 carbon atoms, for example ethylene oxide, propylene oxide, 1,2-butylene oxide, 2,3-butylene oxide or isobutylene oxide, aliphatic 1,2-alkylene oxides having from 5 to 24 carbon atoms, cycloaliphatic alkylene oxides, for example cyclopentene oxide, cyclohexene oxide or 1,5,9-cyclododecatrienemonoxide, arylaliphatic alkylene oxides, for example styrene oxide.

Preferred substituted alkylene oxides are, for example, epichlorohydrin, epibromohydrin, 2,3-epoxy-1-propanol, 1-allyloxy-2,3-epoxypropane, 2,3-epoxypropyl phenyl ether, 2,3-epoxypropyl isopropyl ether, 2,3-epoxypropyl octyl ether or 2,3-epoxypropyltrimethylammonium chloride.

The process of the present invention is particularly preferably carried out using 1,2-alkyleneoxides having from 2 to 4 carbon atoms, in particular ethylene oxide or propylene oxide.

In a preferred embodiment, the invention therefore provides a process for preparing hydroxyalkyl carboxylates from a carboxylic acid and an alkylene oxide, wherein the alkylene oxide is a 1,2-alkylene oxide having from 2 to 4 carbon atoms.

Very particular preference is given to using ethylene oxide or propylene oxide as alkylene oxide.

According to the present invention, the carboxylic acid and the alkylene oxide are used in a molar ratio of from 1:0.4 to 1:10. A molar ratio of from 1:1 to 1:1.5 is particularly advantageous and a molar ratio of from 1:1.03 to 1:1.2 is especially useful.

If the multimetal cyanide compound has been applied to a solid support or has been shaped to form a shaped body, the carboxylic acid and the alkylene oxide are particularly preferably used in a molar ratio of <1:1.5, more preferably <1:1 and very particularly preferably <1:0.8.

For the purposes of the present invention, the reaction of the carboxylic acid with the alkylene oxide can be carried out at from 20 to 200° C. Preference is given to a temperature range from 40 to 150° C., in particular from 50 to 100° C. The reaction can be carried out either at atmospheric pressure or at subatmospheric pressure, and also at superatmospheric pressure, for example at a pressure of from 0.8 to 50 bar, in particular from 1 to 10 bar.

The invention therefore also provides a process for preparing hydroxyalkyl carboxylates in which the temperature in the reaction of the carboxylic acid with the alkylene oxide is from 50 to 100° C.

In a further embodiment, the invention provides a process for preparing hydroxyalkyl carboxylates in which the pressure in the reaction of the carboxylic acid with the alkylene oxide is from 1 to 10 bar.

According to the present invention it is preferred to carry out the reaction of the carboxylic acid with the alkylene oxide at a temperature from 50 to 100° C. and at a present of from 1 to 10 bar.

In the process of the present invention, the reaction can be carried out as a batch process or continuously. It can be carried out in a stirred reactor, loop reactor, fixed-bed reactor, for example a flat bed catalyst oven, tray reactor, shell-and-tube reactor or full-volume reactor, or in a fluidized-bed reactor, preferably in a shell-and-tube reactor or full-volume reactor, in particular in a full-volume reactor.

In a preferred embodiment, the present invention accordingly provides a process for preparing hydroxyalkyl carboxylates which is carried out continuously. Fixed-bed operation is particularly advantageous for the process of the present invention. Here, the catalyst used is immobilized in the reactor.

For the purposes of the present invention, the reaction can be carried out with complete conversion of at least one of the starting materials. However, it is likewise possible for one or both starting materials to be only partly reacted. It is, for example, possible according to the present invention for the reaction to be carried out so that the conversion of the alkylene oxide is more than 70% and the conversion of the carboxylic acid is more than 40%. However, preference is given to the conversion of the alkylene oxide being more than 85% and the conversion of the carboxylic acid being more than 50%.

If the conversion of the starting materials used is incomplete, the process of the present invention is advantageously carried out with unreacted starting material being separated from the product and being returned to the reactor. The separation is carried out by methods known to those skilled in the art, for example by distillation.

Auxiliaries and additives known to those skilled in the art can be added in the reaction of the carboxylic acid with an alkylene oxide. In a preferred embodiment, the reaction of the carboxylic acid with the alkylene oxide is carried out in the presence of at least one polymerization inhibitor. Examples of polymerization inhibitors which can be used are hydroquinone, hydroquinone monomethyl ether, 2,5-di-t-butylhydroquinone, 2,6-di-t-butyl-p-cresol, nitroso compounds such as isoacryloyl nitrite, nitrosodiphenylamine or N-nitrosocyclohexylhydroxylamine, methylene blue, phenothiazine, tannic acid or diphenylamine. It is also possible for two or more of these polymerization inhibitors to be used in the process of the present invention. The polymerization inhibitors are used in amounts of from 10 to 10,000 ppm, in particular from 100 to 1000 ppm, in each case based on the carboxylic acid used.

Furthermore, small amounts of molecular oxygen or nitrogen monoxide which are not of concern from the point of view of safety can be additionally used in the process of the present invention.

It is not necessary to use solvents for the reaction of the carboxylic acid with the alkylene oxide in the process of the present invention. However, it is likewise possible to carry out the process of the present invention in the presence of water or organic solvents, for example aliphatic, cycloaliphatic or aromatic hydrocarbons, alcohols, ethers, acetals, ketones, esters or cyclic carbonates.

In the process of the present invention, the hydroxyalkyl carboxylate can be isolated by, for example, distillation from the reaction mixture. The catalyst can then be reused in the form of the distillation residue or after separation from the distillation residue. However, it is likewise possible to separate off the catalyst prior to the distillation. It can be separated off by, for example, filtration, in particular deep bed filtration, crossflow filtration, membrane filtration or ultrafiltration.

The hydroxyalkyl carboxylates prepared according to the present invention can be used, for example, as raw materials for surface coatings or as monomers for free-radical homopolymerizations or copolymerizations. The present invention therefore also provides for the use of the hydroxyalkyl carboxylates prepared according to the present invention as raw materials for surface coatings.

The following examples illustrate the invention.

### EXAMPLES

### Pulverulent Catalyst

### Preparative Example 1

(Catalyst Synthesis)

540 g of a 30% strength aqueous solution of iron(III) chloride hexahydrate were added dropwise to 950 g of a 20% strength aqueous solution of potassium hexacyanoferrate(II) trihydrate while stirring. The mixture was stirred for another 30 minutes and subsequently filtered on a suction filter. The filter residue was washed twice with water and once with methanol by stirring an appropriate slurry for 30 minutes in each case and then removing the liquid by suction filtration. After the last suction filtration, the solid was dried at 50° C. under reduced pressure. 178 g of a black powder were obtained.

### Example 1

(Synthesis of 2-hydroxyethyl Acrylate)

In a stirred apparatus fitted with a dry ice condenser, a solution of 15.2 g of catalyst from Example 1 in 504.4 g of acrylic acid stabilized with 200 ppm of hydroquinone monomethyl ether and 500 ppm of phenothiazine were treated under a nitrogen atmosphere enriched with 2% by volume of oxygen with 339 g of gaseous ethylene oxide at 50-70° C. for a period of 1.5 hours. The mixture was stirred for another 3.5 hours. Analysis of the crude product by gas chromatography indicated that it was a mixture comprising 91.3% of 2-hydroxyethyl acrylate, 6.0% of diethylene glycol monoacrylate and 0.5% of triethylene glycol monoacrylate together with 0.5% of acrylic acid.

### Comparative Example 1

Catalyst Synthesis

370 kg of aqueous hexacyanocobaltic acid (cobalt content: 9 g/l of cobalt) were placed in a stirred vessel having a capacity of 800 l and fitted with an oblique blade turbine, immersed tube for metered addition, pH electrode, conductivity cell and scattered light probe and heated to 50° C. while stirring. 209.5 kg of aqueous zinc acetate dihydrate solution (zinc content: 2.7% by weight), which had likewise been heated to 50° C., was subsequently added over a period of 50 minutes while stirring (stirrer power: 1 W/l). 8 kg of Pluronic PE 6200 (BASF AG) and 10.7 kg of water were subsequently added while stirring. 67.5 kg of aqueous zinc acetate dihydrate solution (zinc content: 2.7% by weight) were then metered in over a period of 20 minutes at 50° C. while stirring (stirrer power: 1 W/l). The suspension was stirred at 50° C. until the pH had dropped from 3.7 to 2.7 and remained constant. The suspension obtained in this way was subsequently filtered by means of a filter press and the precipitate was washed with 400 l of water in the filter press.

Ethoxylation of Acrylic Acid:

In a procedure analogous to Example 1, the catalyst prepared by the method described in the comparative example, which was not according to the present invention, was used in place of a catalyst according to the present invention.

The metered addition of only 198 g of ethylene oxide took 3 hours before the ethylene oxide absorption ceased. Analysis by gas chromatography indicated the presence of 45.2% of unreacted acrylic acid together with 43.5% of 2-hydroxyethyl acrylate and, despite the low conversion, 3.7% of diethylene glycol monoacrylate and 0.3% of triethylene glycol monoacrylate.

### Compacted Catalyst

### Preparative Example 2

(Catalyst Synthesis)

100 g of ammonium hexacyanoferrate [NH₄Fe₂(CN)₆] (Manox Iron Blue Easispense HSB 3, Degussa-Hüls) were mixed with 25 g of Secar 80 alumina cement (80% of Al₂O₃, 19% of CaO, traces of SiO₂, FeO, Fe₂O₃, TiO₂, MgO, K₂O, Na₂O, SO₃, Lafarge) and compounded in a kneader by addition of 70 ml of H₂O. After addition of 6.25 g of Walocel (wallpaper paste based on methylcellulose, Wolff Walsrode AG), the mixture was compacted for about 45 minutes and subsequently extruded by means of a ram extruder at a pressure of 40 bar to form 2 mm extrudates. These were allowed to harden overnight in air and were subsequently dried at 120° C. in air for 16 hours.

### Preparative Example 3

(Catalyst Synthesis)

The procedure of Preparative Example 2 was repeated using only 3.75 g of Walocel and, in addition, 1.25 g of Lutexal P (polyammonium acrylate, BASF AG).

### Preparative Example 4

(Catalyst Synthesis)

The procedure of Preparative Example 2 was repeated with 6.25 g of ammonium carbonate being additionally added in the compaction step.

### Preparative Example 5

(Catalyst Synthesis)

The procedure of Preparative Example 2 was repeated with 6.35 g of ammonium hydrogen carbonate being additionally added in the compaction step.

### Example 2

(Preparation of Hydroxvethyl Acrylate)

A tube reactor having a reactor volume of 100 ml was 75% filled with a mixture of 28 g of catalyst from Preparative Example 4 (2 mm extrudates, crushed again) and 66 g of glass spheres and firstly flushed overnight with acrylic acid at a reactor temperature of 50° C. After no polymerization was observed, ethylene oxide (EO) was metered in. The reaction temperature was set to 50-60° C. and the reactor pressure was set to 45 bar. The results obtained are shown in Table 1.

TABLE 1

| | | | | Conver- | | |
|---|---|---|---|---|---|---|
| Ethylene | | | Molar | sion | Conver- | Selec- |
| oxide | Acrylic acid | T | ratio of | of EO | sion | tivity |
| (EO) [g/h] | (AA) [g/h] | [° C.] | EO:AA | [%] | of AA [%] | [%] |
| 20 | 29 | 50 | 1.2 | 80 | 75 | 75 |
| 20 | 29 | 55 | 1.2 | 90 | 75 | 75 |
| 13 | 29 | 55 | 0.7 | 90 | 65 | 96 |

We claim:

1. A process for preparing hydroxyalkyl carboxylates, which comprises reacting at least one carboxylic acid with at least one alkylene oxide in the presence of a multimetal cyanide compound of the formula I as catalyst:
M³_{Z}M¹ₐ[M²(CN)_{b}(A)_{c}]_{d}·fM¹_{g}Xₙ·mM³ₚY_{q} ·h(H₂O)·eL·kP (I)
where
M¹ is at least one metal ion selected from the group consisting of Zn²⁺, Fe²⁺, Fe³⁺, Co³⁺, Ni²⁺, Mn²⁺, Co²⁺, Sn²⁺, Pb²⁺, Mo⁴⁺, Mo⁶⁺, Al³⁺, V⁴⁺, V⁵⁺, Sr²⁺, W⁴⁺, W⁶⁺, Cr²⁺, C³⁺, Cd²⁺, Hg²⁺, Pd²⁺, Pt²⁺, V²⁺, Mg²⁺, Ca²⁺, Ba²⁺, Cu²⁺,
M² is at least one metal ion selected from the group consisting of Fe²⁺, Fe³⁺, Co²⁺, CO³⁺, Mn²⁺, Mn³⁺, V⁴⁺, V⁵⁺, Cr²⁺, Cr³⁺, Rh³⁺, Ru²⁺, Ir³⁺,
M¹ and M² are identical or different and at least M¹ or M² is Fe²⁺ or Fe³⁺,
M³ is at least one metal ion selected from the group consisting of Li⁺, Na⁺, K⁺, Rb⁺, Cs⁺, Mg²⁺, Ca²⁺, Ba²⁺, Sr²⁺, ammonium ions of the formula R¹R²R³R⁴N⁺, where R¹, R², R³ and R⁴ are each H or a hydrocarbon radical having from 1 to 6 carbon atoms,
A, X and Y are each, independently of one another, an anion selected from the group consisting of halide, hydroxide, sulfate, carbonate, cyanide, thiocyanate, isocyanate, cyanate, carboxylate, oxalate, nitrate, nitrosyl, hydrogensulfate, phosphate, dihydrogenphosphate, hydrogenphosphate and hydrogencarbonate,
L is a water-miscible ligand selected from the group consisting of alcohols, aldehydes, ketones, ethers, polyethers, esters, polyesters, polycarbonate, ureas, amides, primary, secondary and tertiary amines, ligands containing pyridine nitrogen, nitriles, sulfides, phosphides, phosphites, phosphines, phosphonates and phosphates,
k is a fraction or integer greater than or equal to zero, and
P is an organic additive,
a, b, c, d, g, n, p, q and z are selected so that the compound (I) is electrically neutral, where c or z or c and z may be 0,
e is the number of ligand molecules and is a fraction or integer greater than 0 or is 0,
f, k, h and m are each, independently of one another, a fraction or integer greater than 0 or are 0, wherein the temperature in the reaction is from 50 to 100° C. or the pressure in the reaction of the carboxylic acid with the alkylene oxide is from 1 to 10 bar or both.

2. A process for preparing hydroxyalkyl carboxylates as claimed in claim 1, wherein the multimetal cyanide compound has been applied to a solid support or has been shaped to form a shaped body.

3. A process for preparing hydroxyalkyl carboxylates as claimed in any of the preceding claims, wherein one or more of the following conditions are fulfilled:
(A) M¹ is selected from the group consisting of Zn²⁺, Fe²⁺, Fe³⁺, Co³⁺, Ni²⁺, Mn²⁺, Co²⁺;
(B) M² is selected from the group consisting of Fe²⁺, Fe³⁺, Co³⁺;
(C) M³ is selected from the group consisting of Na⁺, K⁺, ammonium ions of the formula R¹R²R³R⁴N⁺;
(D) M¹ or M² is Fe²⁺ or Fe³⁺.

4. A process for preparing hydroxyalkyl carboxylates as claimed in any of the preceding claims, wherein both M¹ and M² are Fe²⁺ or Fe³⁺.

5. A process for preparing hydroxyalkyl carboxylates as claimed in any of the preceding claims, wherein the multimetal cyanide compound is crystalline.

6. A process for preparing hydroxyalkyl carboxylates as claimed in any of the preceding claims, wherein the catalyst is used in an amount of from 0.01 to 30% by weight, based on the amount of carboxylic acid.

7. A process for preparing hydroxyalkyl carboxylates as claimed in any of the preceding claims, wherein the carboxylic acid is acrylic acid or methacrylic acid.

8. A process for preparing hydroxyalkyl carboxylates as claimed in any of the preceding claims, wherein the alkylene oxide is ethylene oxide or propylene oxide.

9. A process for preparing hydroxyalkyl carboxylates as claimed in any of the preceding claims which is carried out continuously.
